(19) United States
(12) Patent Application Publication
(10) Pub. No.: US 2005/0124877 A1
(43) Pub. Date: Jun. 9, 2005

(54) Device and method for supporting placement of a therapeutic device in a blood vessel

(76) Inventors: Henry Nita, Redwood Shores, CA (US); Jeff Sarge, Fremont, CA (US)

Correspondence Address: Raymond Sun, 12420 Woodhall Way, Tustin, CA, 92782, US

(21) Appl. No.: 10/730,421

(22) Filed: Dec. 8, 2003

(51) Int. Cl. A61B 6/00

(52) U.S. Cl. 600/434; 604/527

(57) ABSTRACT

A therapeutic system includes a guide catheter having a lumen, a sheath having an elongate body that has a lumen and an angled distal end, with the sheath extending through the lumen of the guide catheter, and a catheter extending through the lumen of the sheath. The sheath can be advanced independently beyond the distal end of the catheter, or retracted proximal from the distal end of the catheter. The sheath can also be torqued to redirect the angled distal end of the sheath.

### BACKGROUND OF THE INVENTION

1. Field of the Invention

The present invention pertains to medical equipment and techniques, and more particularly, to a device and method for supporting a therapeutic device (such as a catheter) during the ablation of obstructions within tubular anatomical structures such as blood vessels.

2. Description of the Prior Art

A number of ultrasound systems and devices have heretofore been proposed for use in ablating or removing obstructive material from blood vessels. Ultrasound catheters have been utilized to ablate various types of obstructions from blood vessels of humans and animals. Successful applications of ultrasound energy to smaller blood vessels, such as the coronary arteries, requires the use of relatively small diameter ultrasound catheters which are sufficiently small and flexible to undergo transluminal advancement through the tortuous vasculature of the aortic arch and coronary tree.

A common problem experienced by these ultrasound catheters relates to the need for accurately positioning the ultrasound device inside a patient's vasculature, and in particular, where the vasculature contains smaller and more tortuous vessels. For example, guide catheters are typically used to assist therapeutic devices (such as angioplasty catheters, including ultrasound catheters) in accessing either left or right coronary artery sites. The guide catheters provide support for these catheters, not only during the access, but also during a therapeutic procedure. Maneuvering of therapeutic catheters and placement of these catheters at a treatment site (e.g., a lesion) is usually performed under fluoroscopy with dye injections to assess and observe the placement. During this maneuvering and placement, the catheter must be able to traverse tortuous pathways through blood vessels in the least traumatic manner possible.

Accurate placement of therapeutic catheters is very important from a safety perspective and helps to avoid perforations, dissections, and other unwanted MACE events. Accurate placement of therapeutic catheters is also important for efficacy and procedure time.

Conventional guide catheters cannot help to facilitate accurate placement of a catheter at a specific location in a blood vessel. Accurate placement sometimes requires that the catheter be rotated and manipulated when inside a vessel. However, it is often not possible to accurately place a catheter at a desired location by manipulating the catheter only. The guide catheter manipulations to facilitate the catheter placement are basically impossible due to its ostial engagement. Any manipulation of the guide catheter will cause the guide catheter to disengage from the ostium.

Thus, there still exists a need for a device and a method for facilitating accurate placement of a therapeutic device (such as a catheter) at a specific location in a blood vessel.

### SUMMARY OF THE DISCLOSURE

It is an object of the present invention to provide a device for facilitating accurate placement of a therapeutic device at a specific location in a blood vessel.

It is another object of the present invention to provide a method for accurate placement of a therapeutic device at a specific location in a blood vessel.

It is yet another object of the present invention to provide a device for supporting the placement of an ultrasound catheter inside a blood vessel.

In order to accomplish the objects of the present invention, there is provided a therapeutic system that includes a guide catheter having a lumen, a sheath having an elongate body that has a lumen and an angled distal end, with the sheath extending through the lumen of the guide catheter, and a catheter extending through the lumen of the sheath. The sheath can be advanced independently beyond the distal end of the catheter, or retracted proximal from the distal end of the catheter. The sheath can also be torqued to redirect the angled distal end of the sheath.

### BRIEF DESCRIPTION OF THE DRAWINGS

FIG. 1 is a perspective view of a therapeutic ultrasound system according to the present invention.

FIG. 2 is a perspective view of an intermediate sheath that can be used to support the placement of an ultrasound catheter of the system of FIG. 1.

FIG. 3 is a cross-sectional view of the intermediate sheath of FIG. 2.

FIG. 4A illustrates how a catheter is placed in a conventional procedure.

FIGS. 4B-4E illustrate how the sheath of the present invention supports the placement of an ultrasound catheter of the system of FIG. 1.

FIG. 5A is an exploded cross-sectional view of the valved fitting of the system of FIG. 1.

FIG. 5B is an assembled cross-sectional view of the valved fitting of the system of FIG. 1.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims. In certain instances, detailed descriptions of well-known devices, compositions, components, mechanisms and methods are omitted so as to not obscure the description of the present invention with unnecessary detail.

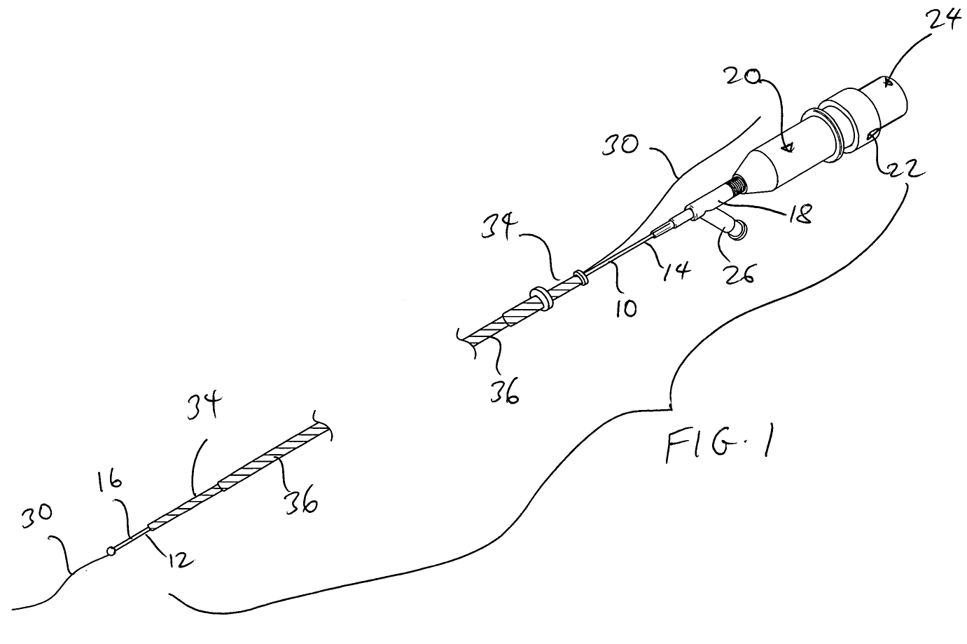

FIG. 1 illustrates a therapeutic ultrasound system according to the present invention for use in ablating and removing occlusive material inside the vessel of an animal or human being. The ultrasound system includes an ultrasonic catheter device 10 which has an elongate catheter body having a proximal end 14, a distal end 16, and defining at least one main lumen (not shown) extending longitudinally therethrough. The ultrasound catheter device 10 is operatively coupled at its proximal end 14, by way of a Y-connector 18, a catheter knob 20 and a slide collar 22, to an ultrasound transducer (not shown) which is housed inside a transducer housing 24. The ultrasound transducer is connected to a signal generator (not shown), which sends an electrical signal to the ultrasound transducer. The ultrasound transducer converts the electrical signal to ultrasound energy, which subsequently passes through the catheter device 10 and is delivered to the distal end 16. Extending longitudinally through the main lumen is an elongate ultrasound transmission member (not shown) having a proximal end which is removably connectable to the ultrasound transducer such that ultrasound energy will pass through the ultrasound transmission member. As such, when the signal generator is actuated, ultrasound energy will pass through the ultrasound transmission member to the distal end 16 of the catheter body. A guidewire 30 may be utilized in conjunction with the catheter device 10.

The ultrasound catheter device 10 can be embodied in the form of any known ultrasound catheter, and will not be described in greater detail herein. Examples of catheter devices 10 that can be utilized in the present invention are illustrated in U.S. Ser. No. 10/211,418, filed Aug. 2, 2002, and entitled “Therapeutic Ultrasound System”, and U.S. Ser. No. 10/601,245, filed Jun. 20, 2003, and entitled “Therapeutic Ultrasound System”, whose disclosures are incorporated by this reference as though set forth fully herein.

The frontal portion of the Y-connector 18 is connected to the proximal end 14 of the catheter 10 using techniques that are well-known in the catheter art. An injection pump (not shown) or IV bag (not shown) or syringe (not shown) can be connected, by way of an infusion tube, to an infusion port or sidearm 26 of the Y-connector 18. The injection pump can be used to infuse coolant fluid (e.g., 0.9% NaCl solution) into and/or through the main lumen of the catheter 10. Such flow of coolant fluid may be utilized to prevent overheating of the ultrasound transmission member extending longitudinally through the main lumen. Such flow of the coolant fluid through the main lumen of the catheter 10 serves to bathe the outer surface of the ultrasound transmission member, thereby providing for an equilibration of temperature between the coolant fluid and the ultrasound transmission member. Thus, the temperature and/or flow rate of coolant fluid may be adjusted to provide adequate cooling and/or other temperature control of the ultrasound transmission member. For example, the coolant temperature at the distal end 16 of the catheter 10 is preferably in the range of 35-45 degrees Celsius, and is preferably less than 50 degrees Celsius, since tissue de-naturalization normally occurs above 50 degrees Celsius.

In addition to the foregoing, the injection pump or syringe may be utilized to infuse a radiographic contrast medium into the catheter 10 for purposes of imaging, as described in greater detail below. Examples of iodinated radiographic contrast media which may be selectively infused into the catheter 10 via the injection pump are commercially available as Angiovist 370 from Berlex Labs, Wayne, N.J. and Hexabrix from Malinkrodt, St. Louis, Mo.

The proximal end of the Y-connector 18 is attached to the distal end of the catheter knob 20 by threadably engaging the proximal end of the Y-connector 18 inside a threaded distal bore (not shown) at the distal end of the catheter knob 20. The construction of the catheter knob 20 and the transducer housing 24 can be same as that illustrated in U.S. Ser. No. 10/666,459, filed Sep. 19, 2003, and entitled “Connector for Securing Ultrasound Catheter to Transducer”, whose disclosure is incorporated by this reference as though set forth fully herein. For example, a sonic connector assembly is housed inside the catheter knob 20 for effectively connecting the ultrasound transmission member to the transducer in a manner which reduces step sonic amplification and provides a smooth connection transition of the transmission member, thereby reducing the stress and fatigue experienced by the transmission member.

Referring still to FIG. 1, the ultrasound system further includes an intermediate sheath 34 that is adapted to receive the catheter device 10, and a guide catheter 36 that is adapted to receive the intermediate sheath 34 inside the main lumen of the guide catheter. The guide catheter 36 can be any conventional guide catheter, and shall not be described in greater detail herein.

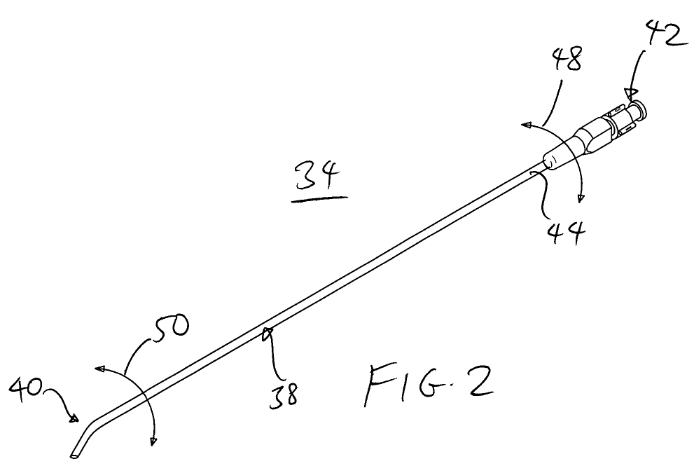

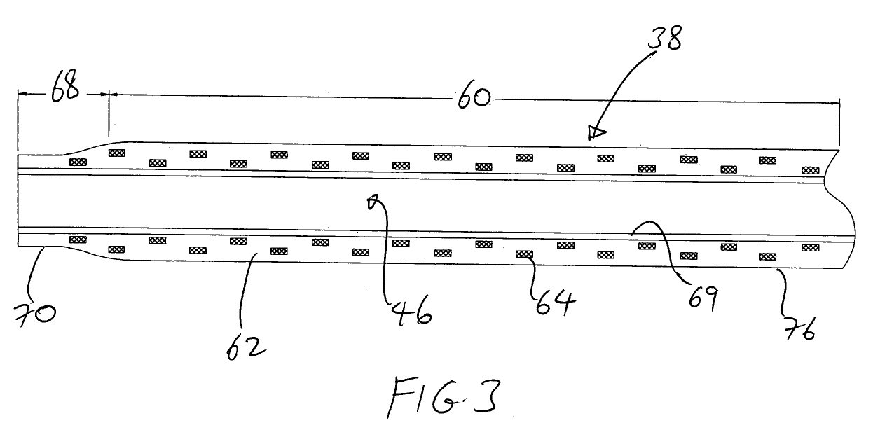

Referring to FIGS. 2 and 3, the sheath 34 has an elongate body 38 with a main lumen 46 extending therethrough for receiving the ultrasound catheter 10. The elongate body 38 has a preshaped angled distal end 40 and a valved fitting 42 provided at the proximal end 44. The distal end 40 is angled by an angle of between 10 degrees and 90 degrees. The angled distal end 40 facilitates the redirection of the ultrasound catheter 10 disposed inside the main lumen 46, which can be controlled by applying a torque input (see arrows 48 in FIG. 2) to the proximal end 44. This torque input is transmitted to a torque output (see arrows 50 in FIG. 2) at the distal end 40. The operation of the sheath 34 will be described in greater detail hereinbelow.

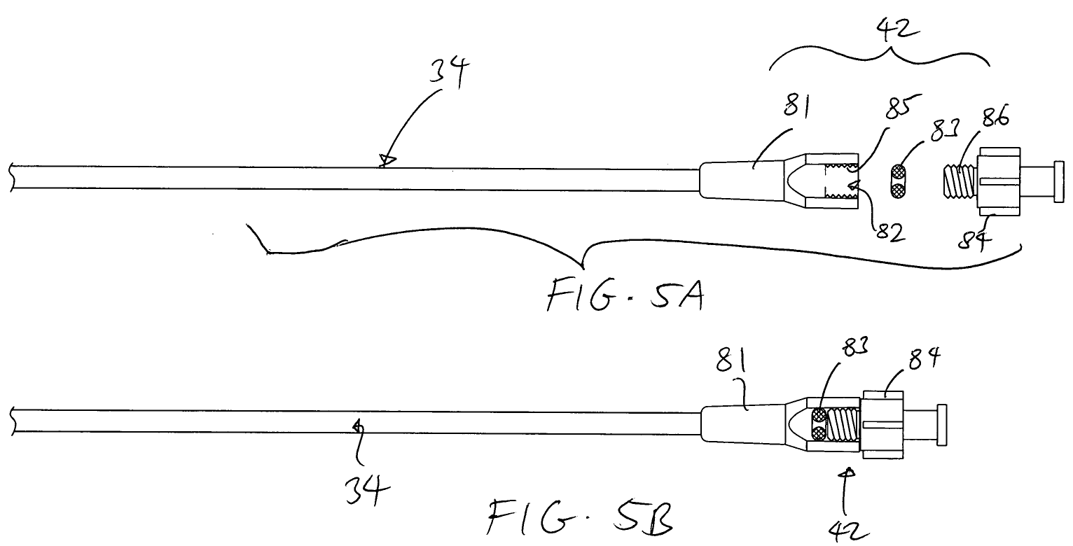

Referring to FIGS. 5A and 5B, the valved fitting 42 is connected to the proximal end 44 of the elongate body 38 by adhesive bond. The valved fitting 42 functions to prevent backflow of blood out of the sheath 34 and around the ultrasound catheter 10 at its proximal end 14. The valved fitting 42 has a distal portion 81 which is connected to the sheath 34 at its proximal end 44 by a conventional glue bond. The distal portion 81 has an inner bore 82. A standard O-ring 83 (which can be made of rubber or silicone) is positioned inside the bore 82. A proximal cup 4 is positioned on the end of the valved fitting 42. The distal portion 81 and the proximal cup 84 are connected together via internal threads 85 positioned inside the bore 82 and external threads 86 positioned on the proximal cup 84.

Threading the distal portion 81 and the proximal cup 84 together will squeeze the O-ring 83, thereby providing a seal around the catheter 10 which is extended through the bore 82. FIG. 5B illustrates the fully assembled sheath 34.

Referring to FIG. 3, the elongate body 38 includes a main shaft member 60 that can be formed of an outer polymeric material 62 having a reinforcing layer 64 embedded therein. The reinforcing layer 64 can be a braid, a coil, a double coil, an opposite wound coil, or the like. The reinforcing layer 64 can be embodied in the form of stainless steel or a superelastic alloy. An inner lubricious polymeric material 69 lines the inner walls of the main lumen 46. The elongate body 38 also includes a distal shaft member 68 extending distal of the main shaft member 60, with the outer diameter of the distal shaft member 68 being smaller than the outer diameter of the main shaft member 60. This is because a smaller diameter provides a lower profile of the sheath 34 on its distal end for better access to a tortuous blood vessel. The distal shaft member 68 can be formed of a polymeric material 70 that is free of any reinforcements. The distal end 40 is angled, with the length of the angled portion being about 5 mm to 30 mm. The hardness of the polymeric material 70 at the distal shaft member 68 can be the same as the hardness of the polymeric material 62 at the main shaft member 60. Examples of the polymeric materials 62, 70 can include, but are not limited to, nylon and urethane. The outer surface of the elongate body 38 can be coated with a lubricious coating 76 to facilitate smooth tracking of the sheath 34 through the lumen of the guide catheter 36 and the vasculature of the patient.

In use, the catheter 10 is positioned inside the lumen 46 of the sheath 34, and the combined catheter 10 and sheath 34 is introduced into the vasculature of a patient over a guide wire 30 through a conventional guide catheter 36. As the combined catheter 10 and sheath 34 is advanced through the vasculature, the sheath 34 may be independently advanced distally to or beyond the distal tip of the catheter 10 when additional support or redirection is needed. Alternatively, the sheath 34 may be retracted proximal from the distal tip of the catheter 10 if needed.

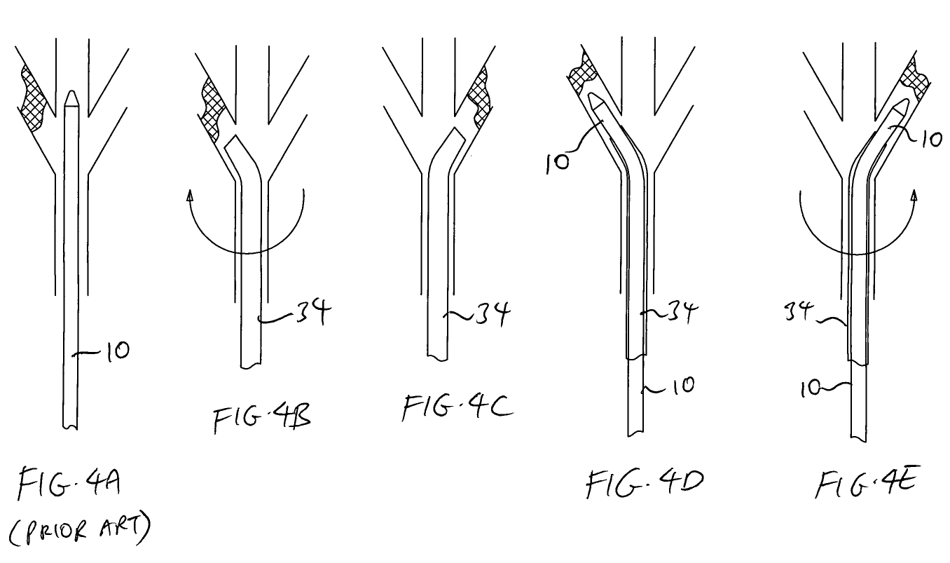

FIGS. 4A-4E illustrate how the sheath 34 can facilitate accurate placement of the catheter 10. FIG. 4A illustrates the conventional placement of a catheter 10 without the sheath 34. In this case, the catheter 10 typically prefers to track into vessels or branches that are straight ahead of it. Therefore, it would be difficult to navigate the catheter 10 into one of the branches shown in FIG. 4A. On the other hand, as shown in FIGS. 4B and 4C, the elongated body 38 of the sheath 34 can be torqued so that the angled distal end 40 can be easily navigated into a branch. Then, as shown in FIGS. 4D and 4E, the catheter 10 can be steered or directed by the enclosing sheath 34 into the desired branch. Here, the sheath 34 can be rotated independently by 360 degrees and its angled distal end 40 positioned in a desired branch location.

Although the present invention is being described in connection with an ultrasound catheter 10, the catheter 10 can be any type of catheter, including but not limited to a balloon angioplasty catheter, an atherectomy catheter, or diagnostic catheters, among others.

While the description above refers to particular embodiments of the present invention, it will be understood that many modifications may be made without departing from the spirit thereof. The accompanying claims are intended to cover such modifications as would fall within the true scope and spirit of the present invention.

1. A therapeutic system, comprising:
a guide catheter having a lumen;
a sheath having an elongate body that has a lumen and a distal end, the sheath extending through the lumen of the guide catheter; and
a catheter extending through the lumen of the sheath.

2. The system of claim 1, wherein the sheath further includes a proximal end, and a valved fitting provided at the proximal end of the sheath.

3. The system of claim 1, wherein the elongate body comprises a main shaft member and a distal shaft member, with the lumen of the sheath extending through the main shaft member and a distal shaft member.

4. The system of claim 3, wherein the main shaft member is formed of an outer polymeric material having a reinforcing layer embedded therein.

5. The system of claim 4, wherein the reinforcing layer is made of stainless steel.

6. The system of claim 4, wherein the reinforcing layer is made of a superelastic alloy.

7. The system of claim 4, wherein the reinforcing layer is a braid.

8. The system of claim 4, wherein the reinforcing layer is a coil.

9. The system of claim 3, wherein the lumen of the sheath has an inner wall, with a lubricious polymeric material provided on the inner wall of the lumen of the sheath.

10. The system of claim 3, wherein the outer diameter of the distal shaft member is smaller than the outer diameter of the main shaft member.

11. The system of claim 3, wherein the distal shaft member is formed of a polymeric material that is free of any reinforcments.

12. The system of claim 3, wherein the hardness of the material at the distal shaft member is equal to the hardness of the material at the main shaft member.

13. The system of claim 1, wherein the elongate body has an outer surface that is coated with a lubricious coating.

14. The system of claim 1, wherein the catheter is an ultrasound catheter.

15. The system of claim 1, wherein the catheter has a proximal end, the system further including a transducer housing coupled to the proximal end of the catheter.

16. The system of claim 1, wherein the distal end of the elongate body is angled by an angle of between 10 degrees and 90 degrees.

17. A method of placing the distal end of a catheter at a desired location inside a vessel, comprising:
providing a sheath having an elongate body that has a lumen and an angled distal end;
extending a catheter through the lumen of the sheath; and
extending the sheath through the lumen of a guide catheter.

18. The method of claim 17, further including:
advancing the sheath independently beyond the distal end of the catheter.

19. The method of claim 17, further including:
retracting the sheath proximal from the distal end of the catheter.

20. The method of claim 17, further including:
torquing the sheath to redirect the angled distal end of the sheath.
